(19) United States
(12) Patent Application Publication
(10) Pub. No.: US 2010/0298743 A1
(43) Pub. Date: Nov. 25, 2010

(54) THERMALLY-ACTIVATED COUPLING ARRANGEMENTS AND METHODS FOR ATTACHING TOOLS TO ULTRASONIC SURGICAL INSTRUMENTS

(75) Inventors: Scott A. Nield, Hamilton, OH (US); David T. Krumanaker, Cincinnati, OH (US); Aaron C. Voegele, Loveland, OH (US); Shan Wan, Mason, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/469,308

(22) Filed: May 20, 2009

(51) Int. Cl. A61N 7/00; A61B 17/00

(52) U.S. Cl. 601/2; 606/1

(57) ABSTRACT

An ultrasonic surgical instrument that has a waveguide that protrudes distally from the handpiece and a surgical tool that is configured to be coupled to the waveguide upon application of thermal energy to the waveguide or the surgical tool.

### FIELD OF THE INVENTION

The present invention generally relates to surgical instruments, and more particularly, to coupling arrangements and methods for attaching a surgical tool to an ultrasonic surgical instrument.

### BACKGROUND

Ultrasonic surgical instruments are used for the safe and effective treatment of many medical conditions. Such instruments commonly include a handpiece that is coupled to an ultrasonic signal generator. The instruments also include an end effector that receives the ultrasonic vibrations. Ultrasonic vibrations, when transmitted to organic tissue at suitable energy levels and using a suitable end effector, may be used to cut, dissect, elevate, cauterize tissue or to separate muscle tissue off bone. Ultrasonic instruments utilizing solid core technology are particularly advantageous because of the amount of ultrasonic energy that may be transmitted from the ultrasonic transducer, through a waveguide, to the surgical end effector. Such instruments may be used for open procedures or minimally invasive procedures, such as endoscopic or laparoscopic procedures, wherein the end effector is passed through a trocar to reach the surgical site.

Typically, ultrasonic vibration is induced in the surgical end effector by electrically exciting a transducer supported in the handpiece. The transducer may be constructed of one or more piezoelectric or magnetostrictive elements. Vibrations generated by the transducer section are transmitted to the surgical end effector via an ultrasonic waveguide that extends from the transducer section to the surgical end effector. The waveguides and end effectors are designed to resonate at the same frequency as the transducer. Therefore, when an end effector is attached to a transducer, the overall system frequency is the same frequency as the transducer itself.

Solid core ultrasonic surgical instruments may be divided into two types, single element end effector devices and multiple-element end effector. Single element end effector devices include instruments such as scalpels, and ball coagulators. The use of multiple-element end effectors such as clamping coagulators includes a mechanism to press tissue against an ultrasonic blade. Ultrasonic clamp coagulators provide an improved ultrasonic surgical instrument for cutting/coagulating tissue, particularly loose and unsupported tissue, wherein the ultrasonic blade is employed in conjunction with a clamp for applying a compressive or biasing force to the tissue, whereby faster coagulation and cutting of the tissue, with less attenuation of blade motion, are achieved. Surgical elevators are instruments used to help facilitate the elevation and removal of soft tissue during surgery. Surgical elevators are generally employed to separate muscle from bone. Cobb or curette type surgical elevators and used in spine surgery, especially to assist in posterior access in removing muscle tissue from bone.

Regardless of the type of end effector employed, the end effector must be effectively coupled to the waveguide. In some devices, the end effector is permanently coupled to the waveguide by, for example, welding. In other arrangements, the end effector is removably coupled to the waveguide by a threaded arrangement. Such end effectors are often supplied with a torque wrench that, when properly used, is designed to ensure that the end effector is attached to the waveguide by an appropriate amount of torque, while avoiding the possibility of damage or device malfunction due to the application of excessive torque to the end effector. Such wrenches may be designed to interface with a distal end or portion of the end effector. In some wrench arrangements, after the wrench is placed on the distal end of the end effector, the clinician applies torque to the wrench until an audible click is heard at which time the wrench may be removed from the end effector.

While the use of such torque wrenches can effectively ensure that an acoustically secure connection is established between the waveguide and the end effector, the torque wrenches may become lost or misplaced during the preparation of the surgical tools and the surgical suite. In addition, the torque wrenches are typically used to detach the end effector from the handpiece which requires the clinician to locate the torque wrench or other tool after the surgical procedure has been completed. Moreover, if the clinician fails to properly use the torque wrench, there is a risk that the connection between the end effector and the waveguide is insufficient to transmit the desired amount of acoustic motion to the end effector for optimum results.

It would be desirable to provide an ultrasonic surgical instrument that overcomes some of the deficiencies of the current instruments and end effector coupling arrangements. Various embodiments of the ultrasonic surgical instruments overcome these deficiencies.

### SUMMARY

In one general aspect, the various embodiments are directed to an ultrasonic surgical instrument that has a handpiece that operably supports at least one ultrasonic transducer. In various embodiments, the surgical instrument comprises a waveguide that protrudes distally from the handpiece and interacts with the ultrasonic transducers. The surgical instrument further includes a surgical tool that has a coupling portion configured to be coupled to a distal end of the waveguide such that upon application of thermal energy to one of the distal end of the waveguide or the coupling portion, the surgical tool is coupled to the waveguide. The surgical tool may be detached from the waveguide by discontinuing the application of the thermal energy.

In accordance with other embodiments of the present invention, there is provided an ultrasonic surgical instrument that has a handpiece that operably supports at least one ultrasonic transducer therein. In various embodiments, the surgical instrument further comprises a waveguide that protrudes distally from the handpiece and interacts with the ultrasonic transducers such that upon activation of the ultrasonic transducers, the waveguide transmits thermal and vibratory energy to a meltable alloy material comprising the distal end portion of the waveguide. The surgical instrument may further include a surgical tool that has a proximal end portion that has a cavity therein that is sized to receive the distal end portion such that upon activation of the at least one ultrasonic transducer when the distal end portion is received within the cavity, the meltable alloy forms a welded connection between the surgical tool and the waveguide.

In accordance with other embodiments of the present invention there is provided an ultrasonic surgical instrument that has a handpiece that operably supports at least one ultrasonic transducer therein. In various embodiments, the surgical instrument includes a waveguide that protrudes distally from the handpiece and interacts with the ultrasonic transducers. The waveguide is fabricated from at least a first material such that at least a distal end portion of the waveguide has a first coefficient of thermal expansion. The surgical instrument further includes a surgical tool that has a proximal end portion. The surgical tool is fabricated from at least a second material such that at least the proximal end portion of the surgical tool has a second coefficient of thermal expansion that is less than the first coefficient of thermal expansion. The proximal end portion of the surgical tool has a cavity for receiving the distal end portion of the waveguide therein. The cavity is sized and shaped relative to the distal end portion of the waveguide such that upon application of thermal energy to the distal end portion when the distal end portion is received in the cavity, the distal end portion expands to retain the distal end portion within the cavity. The retention force is adequate to transmit ultrasonic energy from the transducer to the waveguide.

In accordance with yet other embodiments of the present invention, there is provided a method for coupling a surgical tool to a waveguide of an ultrasonic surgical instrument that has a handpiece that operably supports at least one ultrasonic transducer therein that interacts with the waveguide. In various embodiments, the method comprises positioning a proximal end of the surgical tool in mating contact with a distal end of the waveguide. The method further includes applying a coupling signal to the waveguide and the proximal end of the surgical tool to cause the proximal end of the surgical tool to be coupled to the distal end of the waveguide. In addition, the method may further include discontinuing the application of the coupling signal to permit the surgical tool to be decoupled from the distal end of the waveguide.

In accordance with other embodiments of the present invention, there is provided a method for coupling a surgical tool to a waveguide of an ultrasonic surgical instrument that has a housing that operably supports at least one ultrasonic transducer therein that interacts with the waveguide. In various embodiments, the method comprises positioning a proximal end of the surgical tool in mating contact with a distal end of the waveguide. The method may further include operating the ultrasonic transducers at a predetermined power level that is greater than a normal operating power level for a period of time sufficient to cause at least a portion of the distal end of the waveguide to be welded to the proximal end of the surgical tool and thereafter operating the ultrasonic transducers at the normal operating power to complete a surgical task with the surgical tool.

In accordance with still other embodiments of the present invention, there is provided an ultrasonic surgical instrument that has a handpiece that operably supports at least one ultrasonic transducer therein. In various embodiments, the surgical instrument comprises a waveguide that protrudes distally from the handpiece and interacts with the ultrasonic transducers. The waveguide may have a distal end that has a tool-receiving cavity therein. The surgical instrument may further include a surgical tool that has a proximal end portion that is configured to be received within the tool-receiving cavity in the distal end of the waveguide. The surgical tool may further have a shroud portion that surrounds the proximal end and protrudes proximally therefrom. In addition, the surgical instrument includes a coupling arrangement for selectively retaining the proximal end of the surgical tool within the tool-receiving cavity. In accordance with various embodiments, the coupling arrangement may comprise a locking member that is supported on the distal end of the waveguide adjacent to the cavity. The locking member may be configured to expand from an unexpanded state to an expanded state when ultrasonic energy is applied to the waveguide from the ultrasonic transducers and to return to the unexpanded state when the application of ultrasonic energy is discontinued. The surgical instrument may further include at least one heat generating and cooling unit that is movably coupled to the handpiece and is configured to interact with the locking member. The at least one heat generating and cooling units may be further configured for locking engagement and disengagement with corresponding portions of the shroud.

### BRIEF DESCRIPTION OF THE FIGURES

The novel features of the various embodiments are set forth with particularity in the appended claims. The various embodiments, however, both as to organization and methods of operation, together with further objects and advantages thereof, may best be understood by reference to the following description, taken in conjunction with the accompanying drawings as follows:

FIG. 1 illustrates an ultrasonic system of various embodiments of the present invention;

FIG. 2 illustrates a coupling arrangement embodiment of the present invention for coupling a surgical tool to a waveguide of an ultrasonic surgical instrument;

FIG. 2A illustrates another coupling arrangement embodiment of the present invention for coupling a surgical tool to a waveguide of an ultrasonic surgical instrument;

FIG. 2B illustrates another coupling arrangement embodiment of the present invention for coupling a surgical tool to a waveguide of an ultrasonic surgical instrument;

FIG. 3 illustrates another coupling arrangement embodiment of the present invention for coupling a surgical tool to a waveguide of an ultrasonic surgical instrument;

FIG. 3A is another view of the coupling arrangement of FIG. 3 with a portion of the waveguide in an expanded condition;

FIG. 3B illustrates another coupling arrangement embodiment of the present invention for coupling a surgical tool to a waveguide of an ultrasonic surgical instrument;

FIG. 4 illustrates another coupling arrangement embodiment of the present invention for coupling a surgical tool to a waveguide of an ultrasonic surgical instrument;

FIG. 5 is another view of the coupling arrangement of FIG. 4 with a portion of the surgical tool shroud shown in cross-section and the latch members in unexpanded conditions;

FIG. 6 is another view of the coupling arrangement of FIG. 4 with the latch members thereof in an expanded condition;

FIG. 7 is a side view of another handpiece embodiment of the present invention;

FIG. 8 is an end view of the handpiece of FIG. 7;

FIG. 9 is an end view of another surgical tool embodiment of the present invention;

FIG. 10 is a side view of the surgical tool of FIG. 9;

FIG. 11 illustrates another coupling arrangement embodiment of the present invention for coupling a surgical tool to a waveguide of an ultrasonic instrument with the shroud portion of the surgical tool shown in cross-section;

FIG. 12 is another cross-sectional view of the coupling arrangement of FIG. 11 taken along a different cutting line and showing the latch members thereof in an expanded condition;

FIG. 13 illustrates another coupling arrangement embodiment of the present invention for coupling a surgical tool to a waveguide of an ultrasonic surgical instrument;

FIG. 14 illustrates one form of a power vs. time curve for the coupling arrangement embodiment depicted in FIG. 13;

FIG. 15 is a partial exploded assembly view of a handpiece and surgical tool embodiment of the present invention with a portion of the handpiece and a portion of the surgical tool shown in cross-section;

FIG. 16 is another partial cross-sectional exploded assembly view of the handpiece and surgical tool of FIG. 16 in a coupling orientation;

FIG. 17 another partial cross-sectional exploded assembly view of the handpiece and surgical tool of FIGS. 15 and 16 in a coupled orientation;

FIG. 18 is a cross-sectional view of a heating and cooling unit of the coupling arrangement embodiment depicted in FIGS. 15-17;

FIG. 19 is another cross-sectional view of the heating and cooling unit of FIG. 18 as the shroud of the surgical tool is being installed thereon;

FIG. 20 is another cross-sectional view of the heating and cooling unit of FIGS. 18 and 19 with the shroud in retaining engagement therewith;

FIG. 21 illustrates an ultrasonic system of various embodiments of the present invention;

FIG. 22 illustrates a coupling arrangement embodiment of the present invention for coupling a surgical tool to a waveguide of an ultrasonic surgical instrument, wherein the distal end of the waveguide is in retaining engagement with the surgical tool; and

FIG. 23 illustrates the coupling arrangement embodiment depicted in FIG. 22 prior to expanding the distal end of the waveguide.

### DETAILED DESCRIPTION

Before explaining the various embodiments in detail, it should be noted that the embodiments are not limited in their application or use to the details of construction and arrangement of parts illustrated in the accompanying Drawings and Description. The illustrative embodiments may be implemented or incorporated in other embodiments, variations and modifications, and may be practiced or carried out in various ways. For example, the surgical instruments and surgical tool configurations disclosed below are illustrative only and not meant to limit the scope or application thereof. Furthermore, unless otherwise indicated, the terms and expressions employed herein have been chosen for the purpose of describing the illustrative embodiments for the convenience of the reader and are not to limit the scope thereof.

The various embodiments relate, in general, to ultrasonic surgical instruments and, more particularly, to coupling arrangements for coupling a surgical tool to the source of ultrasonic energy in such instruments. Examples of ultrasonic surgical instruments are disclosed in U.S. Pat. Nos. 5,322,055 and 5,954,736 and in combination with ultrasonic blades and surgical instruments disclosed in U.S. Pat. Nos. 6,309,400 B2, 6,278,218B1, 6,283,981 B1, and 6,325,811 B1, for example, are incorporated herein by reference in their respective entireties. Also incorporated by reference in their respective entireties are commonly-owned, co-pending U.S. patent application Ser. No. 11/726,625, entitled ULTRASONIC SURGICAL INSTRUMENTS, filed on Mar. 22, 2007 as well as commonly-owned U.S. patent application entitled COUPLING ARRANGEMENTS AND METHODS FOR ATTACHING TOOLS TO ULTRASONIC SURGICAL INSTRUMENTS, Docket No. END6494USNP/080595, filed on even date herewith.

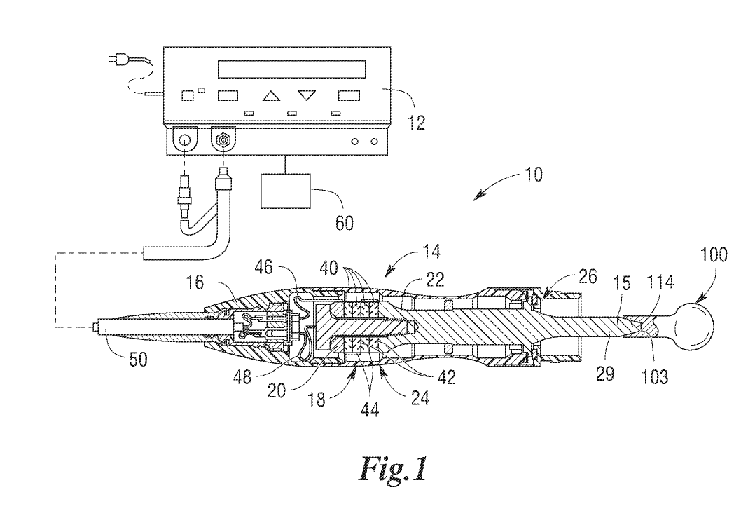

FIG. 1 illustrates an ultrasonic system 10 comprising an ultrasonic signal generator 12 with ultrasonic transducer 14, handpiece 16, and surgical tool 100 which may be employed in accordance with various embodiments of the present invention. Various aspects of such a system are described in further detail in U.S. Patent Publication No. US 2008/0234709 A1, the disclosure of which is herein incorporated by reference in its entirety. The ultrasonic transducer 14, which is known as a “Langevin stack”, may generally include a transduction portion 18, a first resonator or end-bell 20, and a second resonator or fore-bell 22, and ancillary components. The ultrasonic transducer 14 is preferably an integral number of one-half system wavelengths (nλ/2). An acoustic assembly 24 may include the ultrasonic transducer 14, mount 26, and velocity transformer 28.

The distal end of end-bell 20 is connected to the proximal end of transduction portion 18, and the proximal end of fore-bell 22 is connected to the distal end of transduction portion 18. Fore-bell 22 and end-bell 20 have a length determined by a number of variables, including the thickness of the transduction portion 18, the density and modulus of elasticity of the material used to manufacture end-bell 20 and fore-bell 22, and the resonant frequency of the ultrasonic transducer 14.

The transducer may be constructed of one or more piezoelectric or magnetostrictive elements in the instrument handpiece 16. Ultrasonic vibration is induced in the surgical tool 100 by, for example, electrically exciting a transducer which may be constructed of one or more piezoelectric or magnetostrictive elements in the instrument hand-piece. Vibrations generated by the transducer section are transmitted to the surgical tool 100 via an ultrasonic waveguide 28 extending from the transducer section to the surgical tool 100.

In the illustrated embodiment, the transducer is constructed with piezoelectric elements 40. The piezoelectric elements 40 may be fabricated from any suitable material, such as, for example, lead zirconate-titanate, lead meta-niobate, lead titanate, or other piezoelectric crystal material. Each of the positive electrodes 42, negative electrodes 44, and piezoelectric elements 40 has a bore extending through the center. The positive and negative electrodes 42 and 44 are electrically coupled to wires 46 and 48, respectively. Wires 46 and 48 are encased within cable 50 and electrically connectable to ultrasonic signal generator 12 of ultrasonic system 10.

Ultrasonic transducer 14 of the acoustic assembly 24 converts the electrical signal from ultrasonic signal generator 12 into mechanical energy that results in primarily longitudinal vibratory motion of the ultrasonic transducer 14 and surgical tool 100 at ultrasonic frequencies. A suitable generator is available as model number GEN04, from Ethicon Endo-Surgery, Inc., Cincinnati, Ohio. When the acoustic assembly 24 is energized, a vibratory motion standing wave is generated through the acoustic assembly 24. The amplitude of the vibratory motion at any point along the acoustic assembly 24 may depend upon the location along the acoustic assembly 24 at which the vibratory motion is measured. A minimum or zero crossing in the vibratory motion standing wave is generally referred to as a node (i.e., where motion is usually minimal), and an absolute value maximum or peak in the standing wave is generally referred to as an anti-node. The distance between an anti-node and its nearest node is one-quarter wavelength (λ/4).

Wires 46 and 48 transmit the electrical signal from the ultrasonic signal generator 12 to positive electrodes 42 and negative electrodes 44. The piezoelectric elements 40 are energized by an electrical signal supplied from the ultrasonic signal generator 12 in response to a foot switch 60 to produce an acoustic standing wave in the acoustic assembly 24. The electrical signal causes disturbances in the piezoelectric elements 40 in the form of repeated small displacements resulting in large compression forces within the material. The repeated small displacements cause the piezoelectric elements 40 to expand and contract in a continuous manner along the axis of the voltage gradient, producing longitudinal waves of ultrasonic energy. The ultrasonic energy is transmitted through the acoustic assembly 24 to the surgical tool 100.

In order for the acoustic assembly 24 to deliver energy to the surgical tool 100, all components of acoustic assembly 24 must be acoustically coupled to the surgical tool 100. The components of the acoustic assembly 24 are preferably acoustically tuned such that the length of any assembly is an integral number of one-half wavelengths (nλ/2), where the wavelength λ is the wavelength of a pre-selected or operating longitudinal vibration drive frequency f_{d} of the acoustic assembly 24, and where n is any positive integer. It is also contemplated that the acoustic assembly 24 may incorporate any suitable arrangement of acoustic elements. As the present Detailed Description proceeds, those of ordinary skill in the art will readily understand that the system 10 described above is but one example of a myriad of ultrasonic surgical systems that may employ various unique and novel advantages of the embodiments of the present invention.

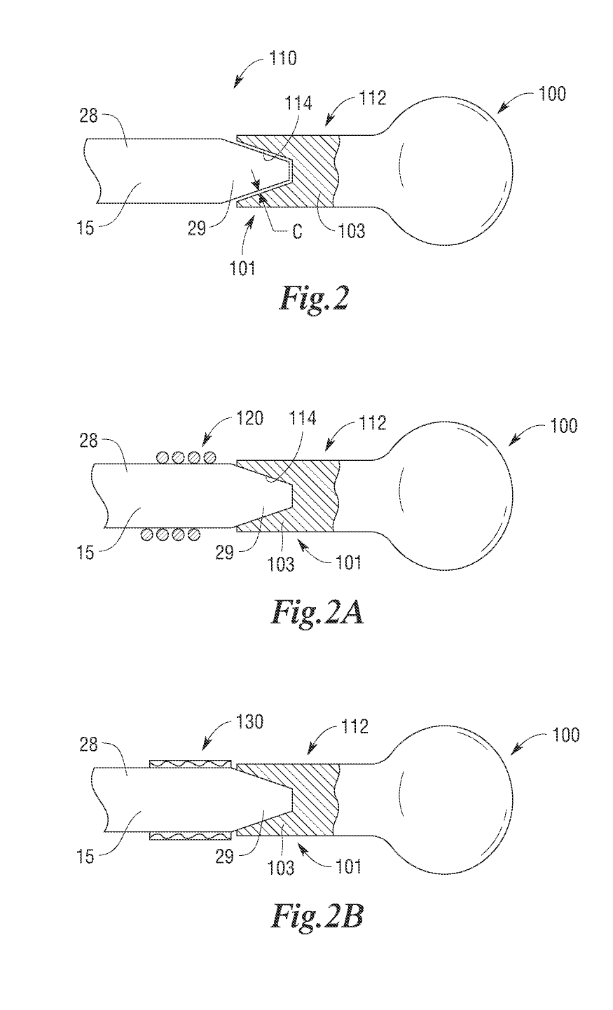

FIGS. 1 and 2 illustrate a coupling arrangement 110 of an embodiment of the present invention for coupling the surgical tool 100 to the waveguide 28. The surgical tool 100 is illustrated as a blade that has a generally smooth exterior surface that is well-suited for coagulation and tissue reshaping applications. However, as used herein, the term “surgical tool” may encompass any surgical end effector or tool or blade that may be operably coupled with an ultrasonic surgical handpiece or other source of ultrasonic energy in a surgical setting and includes, but is not limited to, straight and curved blades, sharp hooks, dissecting hooks, ball coagulators, clamp coagulators, etc. Exemplary blade configurations are described in U.S. Pat. No. 6,423,082 to Houser et al., the disclosure of which is herein incorporated by reference in its entirety. Examples of clamp coagulator arrangements are disclosed in U.S. Pat. No. 6,254,623, the disclosure of which is herein incorporated by reference in its entirety.

In the embodiment depicted in FIGS. 1 and 2, the distal end 29 of the waveguide 28 is configured to be coupled to a coupling portion 101 which, in various embodiments, comprises a complementary-shaped cavity 114 provided in the proximal end portion 112 of the surgical tool 100. For example, the distal end 29 may have a frusto-conical shaped and be sized to be received within cavity 14. The waveguide 28, or at least the distal end portion 29 of the waveguide 28 is fabricated from a first material 15 that has a first coefficient of thermal expansion (CTE1). The surgical tool 100, or at least the proximal end portion 112 of the surgical tool 100, is fabricated from a second material 103 that has a second coefficient of thermal expansion (CTE2) that is less than the first coefficient of thermal expansion. Thus:

CET2<CET1

The distal end portion 29 of the waveguide 28 is sized and shaped relative to the cavity 114 in the proximal end portion 112 of the surgical tool 100 such that a slip fit or an amount of clearance “C” is created between the distal end portion 29 of the waveguide 28 and the cavity 114 when the waveguide 28 and the surgical tool 100 are at approximately the same temperature.

In various embodiments, the waveguide 28, or at least the distal end portion 29 of the waveguide 28, may be fabricated from, for example, aluminum which has a coefficient of thermal expansion of 13.7×10⁻⁶ in/in/degree F. and the proximal end portion 112 of the surgical tool 100 may be fabricated from, for example, titanium which has a coefficient of thermal expansion of 4.34×10⁻⁶ in/in/degree F. In such embodiment, clearance “C” may be approximately 0.0005 inches.

To couple the surgical tool 100 to the waveguide 28, the clinician positions the distal end portion 29 of the waveguide 28 into the cavity 114 of the surgical tool 100 as illustrated in FIG. 2. Thermal energy (i.e., heat) is then applied to the distal end portion 29 of the waveguide 28 to increase the outside diameter or parametrical shape of the distal end portion 29 through thermal expansion. Because CTE1>CTE2, the outer diameter or parametrical shape of the distal end portion 29 of the waveguide 14 will expand to a greater magnitude when compared to the inside diameter or shape of the cavity 114 to establish an interference fit therebetween as illustrated in FIG. 2A. The heat or thermal energy may be applied to the waveguide 28 by a radio frequency (RF) induction coil 120 mounted about the waveguide 28 adjacent the distal end portion 29. In other embodiments, a resistive thermoelectric heat element 130 may be employed. See FIG. 2B. Heat is applied until a sufficient interference fit is established between the proximal end portion 112 of the surgical tool 100 and the distal end portion 29 of the waveguide 28. Thereafter, the heat applicator 120, 130 must continue to be energized to maintain the interference fit throughout use. After the surgical procedure has been completed, the heat applicator 120, 130 may be de-energized. Once the temperature of the proximal end portion 29 of the waveguide 28 returns to the approximate temperature of the proximal end 114 of the surgical tool 100, the surgical tool may be detached from the waveguide 28.

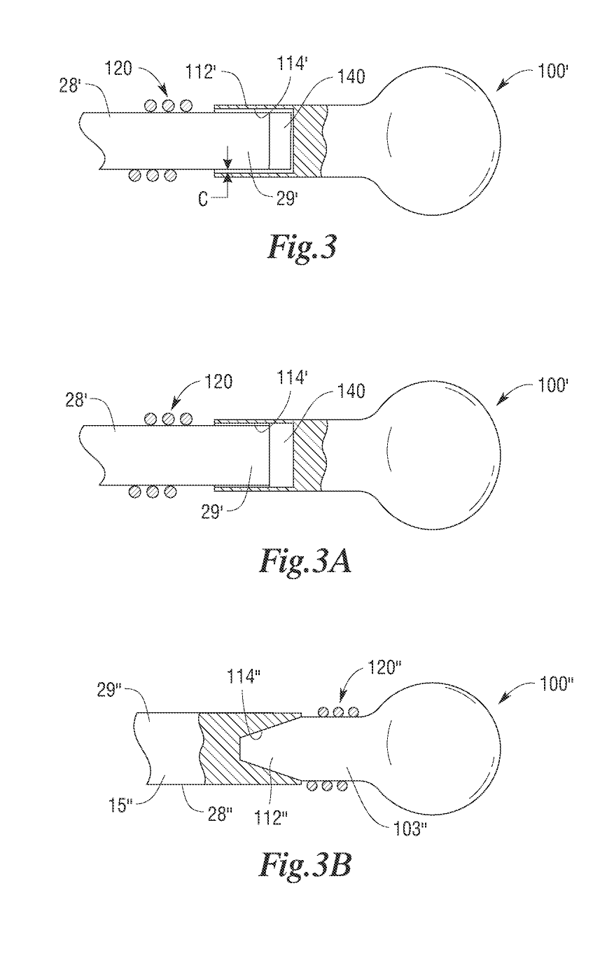

In the embodiment of FIG. 3, the distal end portion 29′ of the waveguide 28′ has a portion 140 that is fabricated from material that has a high coefficient of thermal expansion. For example, the portion 140 may be fabricated from, for example, aluminum, while the remaining portion of the waveguide 28′ may be fabricated from steel. At normal room temperature (i.e., in an unheated state), the portion 140 may have the same diameter or other parametrical shape as the distal end portion 29′ of the waveguide 28′ to enable the portions 29′, 140 to be inserted into the cavity 114′ in the proximal end portion 112′ of the surgical tool 100′. Thus, a predetermined amount of clearance “C” is provided between the portion 140 and the wall of the cavity 114′, prior to the application of heat or thermal energy to the distal end portion 29′ by the heat applicator 120 or 130 (whichever the case may be). To couple the surgical tool 100′ to the waveguide 28′, the heat applicator 120 or 130 is energized to cause portion 140 to expand at a greater rate than the distal end portion 112′ of the surgical tool 100′ to create an interference fit therebetween. See FIG. 3A.

FIG. 3B illustrates an alternative embodiment wherein a cavity 114″ is provided in the distal end 29″ of the waveguide 28″ and the proximal end portion 112″ of the surgical tool 100″ is sized to be received within the cavity 114″. In this embodiment, the distal end 29″ of the waveguide 28″ is fabricated from a first material 15″ that has first coefficient of thermal expansion (CTE1) and the proximal end portion 112″ of the surgical tool 100″ is fabricated from a second material 103″ that has a second coefficient of thermal expansion (CTE2) that is greater than the first coefficient of thermal expansion. Thus:

CET2>CET1

To couple the surgical tool 100″ to the waveguide 28″, the clinician positions the proximal end portion 112″ of the surgical tool 100″ in the cavity 114″ in the distal end portion 29″ of the waveguide 28″ as illustrated in FIG. 3B. Thermal energy (i.e., heat) is then applied to the proximal end portion 112″ of the surgical tool 100″ to increase the outside diameter or parametrical shape of the proximal end portion 112″ through thermal expansion. Because CTE1<CTE2, the outer diameter or parametrical shape of the proximal end portion 112″ of the surgical tool 100″ will expand at a greater magnitude when compared to the inside diameter or shape of the cavity 114″ to establish an interference fit therebetween. The heat or thermal energy may be applied to the proximal end portion 112″ of the surgical tool 100″ by a heat applicator 120 which may comprise a radio frequency (RF) induction coil or resistive heater 120″ mounted on the proximal end portion 112″. Power may be supplied thereto from the handpiece through appropriate connections. For example, the heating element 120″ may be mounted on the surgical tool 100″ and, when the proximal end portion 112″ of the surgical tool 100″ is inserted into the cavity 114″, the heat applicator 120″ may be coupled to wires (not shown) protruding from the handpiece to supply power to the heat applicator 120″. Heat is applied until a sufficient interference fit is established between the proximal end portion 112″ of the surgical tool 100″ and the distal end portion 29″ of the waveguide 28″. Thereafter, the heat applicator 120 may be de-energized and/or removed and the system may be used. Once the temperature of the proximal end portion 112″ of the surgical tool 100″ returns to the approximate temperature of the distal end portion 29″ of the waveguide 28″, the surgical tool 100″ may be detached from the waveguide 28″.

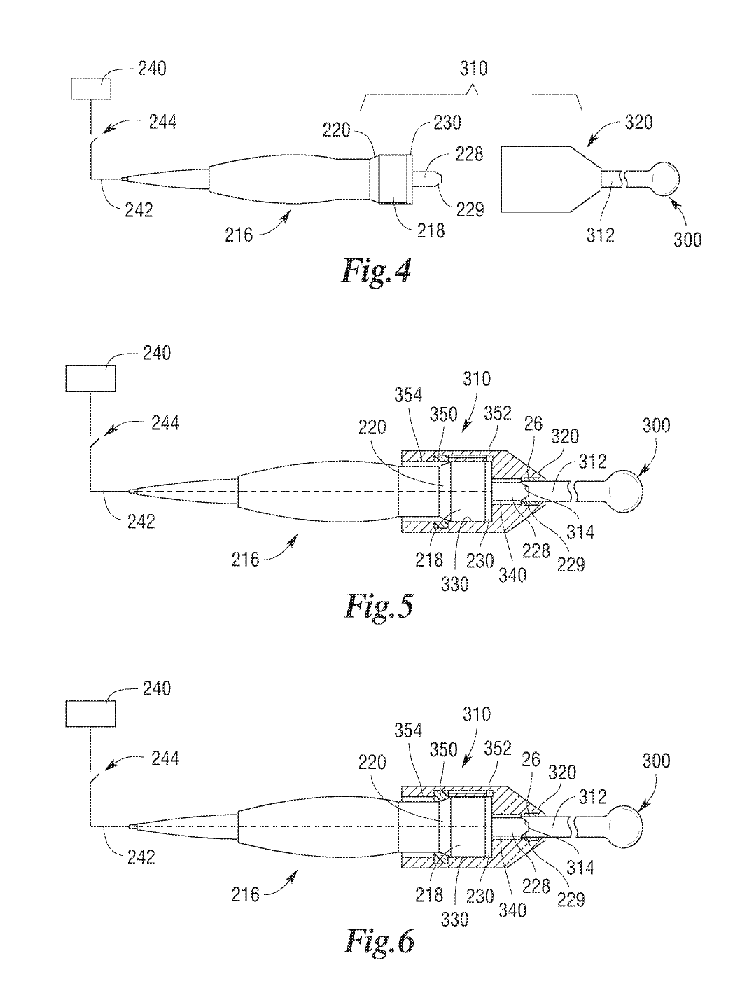

FIGS. 4-6 illustrate use of another coupling arrangement 310 of various embodiments of the present invention for removably coupling a reusable surgical tool 300 to a waveguide 228 of a handpiece 216 that is similar in construction and operation as the aforementioned handpiece 16 except for the differences noted below. In some embodiments, for example, the distal end 229 of the waveguide 228 may be tapered or frusto-conically shaped for receipt within a complementary-shaped cavity 314 provided in a proximal end portion 312 of the surgical tool 300. In this embodiment, the surgical tool 300 includes a housing or shroud portion 320 that supports the distal end portion 312 therein. In various embodiments, the distal end portion 312 may be supported within a mount 26 that facilitates acoustic excitement of the distal end portion 312 relative to the shroud 320. As can be seen in FIGS. 5 and 6, the shroud 320 has a cavity 330 therein for receiving the distal end portion 218 of the handpiece 216 therein. Shroud 320 further has an axial passage 332 to enable the waveguide 228 to extend therethrough into engagement with the proximal end portion 312 of the surgical tool 300. As can be further seen in FIGS. 4-6, the distal end portion 218 of the handpiece 216 has a tapered portion 220 formed thereon. When the distal end portion 218 is received within the cavity 330 of the shroud 320, the tapered portion 220 coincides with at least one selectively expandable latch member 350 mounted in the shroud 320. The latch member(s) 350 may be fabricated from, for example, a shape memory alloy (SMA) and be coupled to corresponding tool contact(s) 352 mounted within the shroud 320. For example, a latch member 350 may be fabricated in the shape of a ring or a hoop from NiTi (Nickel-Titanium), CuZnAl, CuAlNi, etc. and be coupled to contact 352 by contact strip or strips 354. As can also be seen in FIGS. 4-6, an activation contact 230 is mounted in the distal end portion 218 of the handpiece 216. In various embodiments, the activation contact 230 may comprise an annular ring or ring segment(s) formed from electrically conductive material (e.g., berillium copper) and which is in electrical communication (e.g., wired) to a source of electrical power 240. The source of electrical power 240 may comprise, for example, a battery or a source of alternating current and may be integrated with the aforementioned generator arrangement.

FIGS. 5 and 6 illustrate a method of coupling of the surgical tool 300 to the waveguide 228 of the handpiece 216. To initiate the coupling process, the distal end portion 218 of the handpiece is inserted into the cavity 330 in the housing 320 of the surgical tool 300 as shown in FIG. 5 such that activation contact 230 makes electrical contact with tool contact 352 to thereby permit electrical current (actuation signal) to energize the latch member(s) 350. In various embodiments, a switch 244 may be provided in the electric line/wire 242 coupling the actuation contact 230 to the source of electrical power 240. The switch 244 may, for example, be located on the handpiece or the generator. Thus, when the surgical tool 300 is coupled to the waveguide 228 as shown in FIG. 5, and the switch 244 is activated, the latch 350 will be energized and start to expand against the tapered portion 220. Those of ordinary skill in the art will appreciate that the engagement of the latch 350 with the tapered portion 220 causes the tool 300 to be pulled into retaining engagement with the waveguide 228 to achieve an acoustically sufficient connection between the distal end portion 229 of the waveguide 228 and the proximal end portion 312 of the surgical tool 300. See FIG. 6.

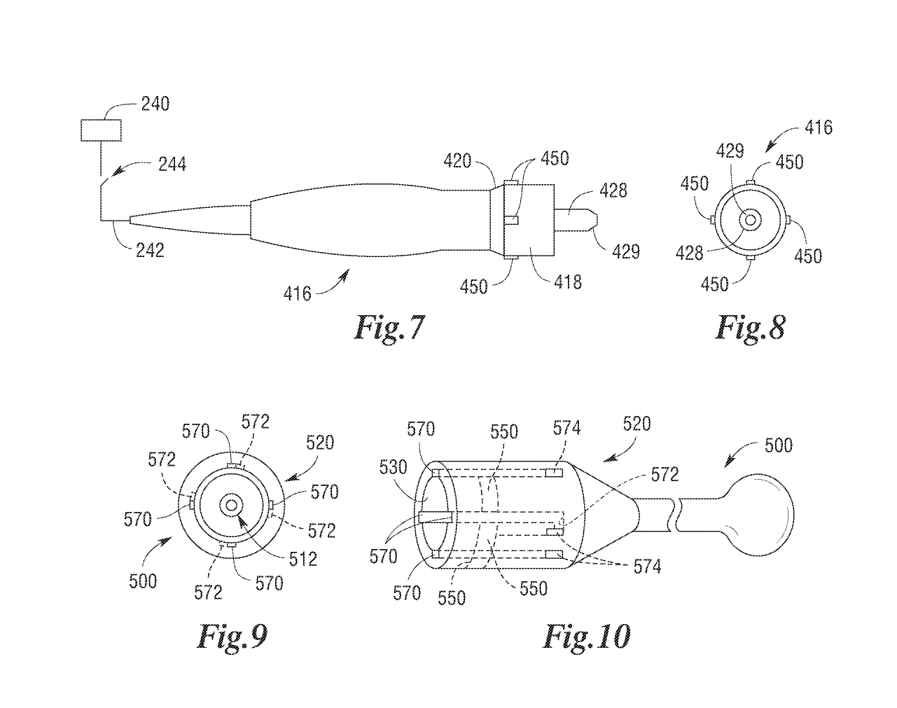

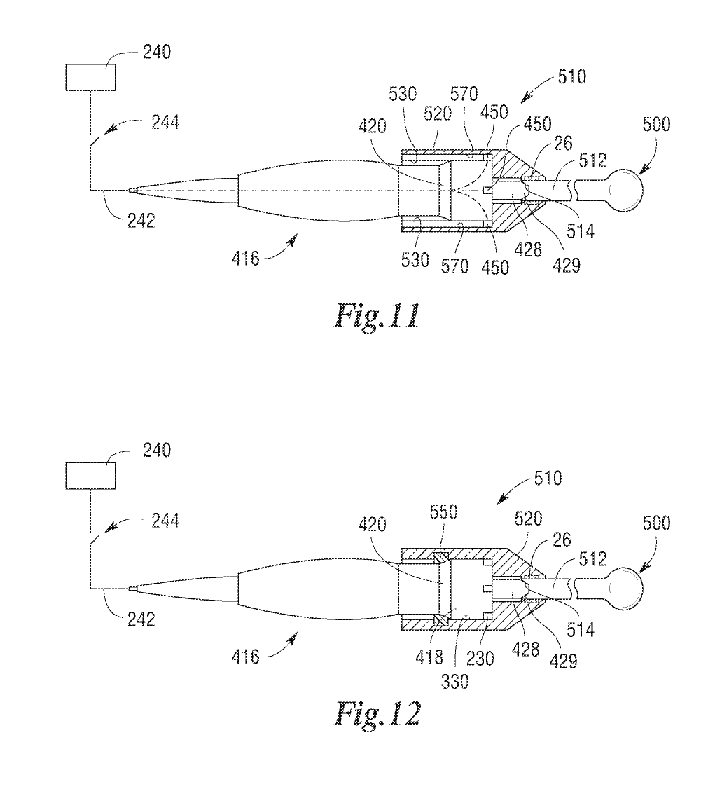

FIGS. 7-12 illustrate another coupling arrangement 510 of various embodiments of the present invention for removably coupling a reusable surgical tool 500 to a waveguide 428 of a handpiece 416 that is similar in construction and operation as the aforementioned handpiece 216 except for the differences noted below. In this embodiment, at least one, and preferably four, contact tabs 450 protrude out of the distal end 418 of the handpiece as shown in FIGS. 7 and 8. One or more of the contact tabs 450 are wired to a source of electrical energy 240. As with the other embodiments, a switch 244 may be provided to control the flow of current from the source 240 to the contact tabs 450. The tool 500 has a tool shroud 520 that has corresponding tab slots 570 therein that are adapted to receive a corresponding one of the contact tabs 450 to enable the shroud 520 to be slid onto the handpiece 416 to the position shown in FIG. 11. Thereafter, the clinician rotates the shroud 520 relative to the handpiece 416 to cause the contact tabs 450 to each be received in a corresponding locking pocket 572 at the end of each slot 570. An electrical contact 574 may be positioned within or adjacent to each locking pocket 572 such that it makes electrical contact with the corresponding contact tab 450 when seated within the locking pocket 572. The electrical contact 574 is in electrical communication with a corresponding one or more expandable latch member segments 550 supported within the shroud 520. The latch member segments 550 are located such that when the tool 500 is seated onto the handpiece and the contact tabs 450 are received in their respect lock pockets 572, the latch member segments 550 are positioned to engage the tapered position 420 of the handpiece 416.

To initiate the coupling process, the distal end portion 418 of the handpiece is inserted into the cavity 530 in the shroud 520 of the surgical tool 500 as shown in FIG. 11 such that the contact tabs 450 are received in their corresponding slots 570, the clinician rotates the handpiece 416 relative to the surgical tool 500 to cause the contact tabs 450 to be seated in their corresponding locking pockets 572 and are in contact with the corresponding electrical contact 574 therein. If the switch 244 is closed, electrical current is then permitted to flow through the electric contacts 574 to the expandable latch member segments 550. As current flows to the expandable latch member segments 550, the latch member segments 550 expand and pull the proximal end 512 of the tool 500 into retaining engagement with the distal end 429 of the waveguide 428.

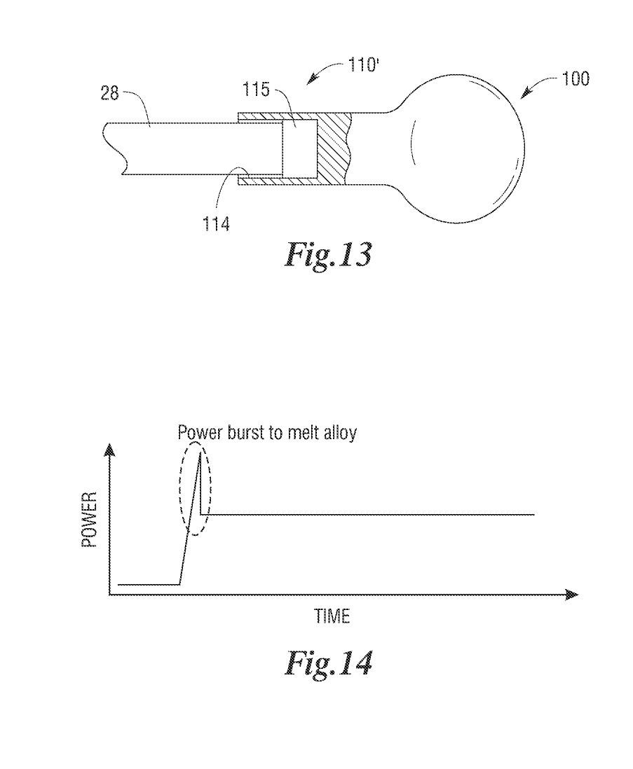

FIGS. 13 and 14 illustrate another coupling arrangement 110′ of various embodiments of the present invention for permanently coupling a surgical tool 100 to a waveguide 28 of a handpiece 16. In this embodiment, the distal end 29 of the waveguide 28 is sized to be received within a cavity 114 in proximal end portion 114 of the surgical tool 100. Positioned within the cavity 114 is some meltable alloy material 115. In various embodiments, the meltable alloy material may comprise, for example, copper-aluminum. In this embodiment, the clinician inserts the distal end 29 of the waveguide 28 into the cavity 114 such that it contacts the meltable alloy material 115. The clinician then operates the generator 12 to provide the waveguide 28 with a sufficient power burst that is sufficient in magnitude and duration to cause the meltable alloy material 115 to weld the waveguide 28 to the tool 100. As illustrated in FIG. 14, once welding is complete, the clinician reduces the power to the normal operating magnitude. For example, normal power magnitude may be 5 watts. To cause the meltable material 115 to sufficiently weld the waveguide 28 to the tool 100, the clinician may have to increase the power to, for example, 50 watts, for approximately 5 seconds (time). The magnitude and duration of such increase may be dependent upon the type of meltable material 115 employed and the transducer arrangement. In each case, however, the magnitude and duration of the power increase should be less than a magnitude and duration that would ultimately result in damage to the transducers or other components of the system.

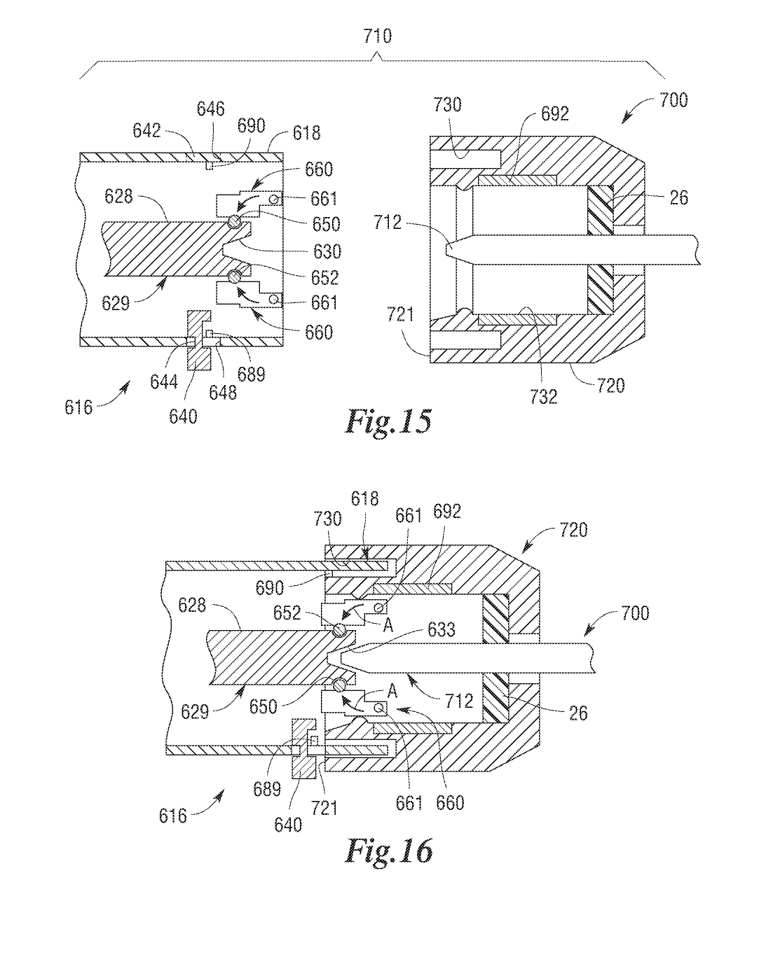

Another coupling arrangement 710 is illustrated in FIGS. 15-20 for removably coupling a reusable surgical tool 700 to a waveguide 628 of a handpiece 616 that is similar in construction and operation as the aforementioned handpiece 16 except for the differences noted below. For example, the distal end 629 of the waveguide 628 may have a frusto-conically shaped cavity 630 therein for receiving a complementary-shaped proximal end portion 712 of a surgical tool 700. In this embodiment, the surgical tool 700 includes a shroud 720 that supports the proximal end portion 712 therein. In various embodiments, the shroud 720 may be fabricated from, for example, Titanium 64 and proximal end portion 712 may be supported within a mount 26 that facilitates acoustically-generated movement of the proximal end portion 712 relative to the shroud 720. As can be seen in FIGS. 15 and 16, the shroud 720 has an annular cavity 730 therein for receiving the distal end portion 618 of the handpiece 616 therein. Shroud 720 further has an axial passage 732 to enable the waveguide 628 to extend therethrough into engagement with the proximal end portion 712 of the surgical tool 700.

As illustrated in FIGS. 15 and 16, the distal end 618 of the handpiece 616 movably supports a release ring 640 that has diametrically opposing stem portions 642, 644 that extend through corresponding slots 646, 648, respectively in the distal end portion 618 of the handpiece 616. The purpose of the release ring 640 will be explained in further detail below. As can also be seen in FIGS. 15 and 16, the distal end portion 629 of the waveguide 628 is also fitted with an annular groove 650 that is configured to receive a locking ring 652 therein. Locking ring 652 may be fabricated from a shape memory alloy (SMA) such as, for example, NiTi (Nickel-Titanium), CuZnAl, CuAlNi, etc. Locking ring 652 may also be supported in at least two, and preferably four, heat generating and cooling units 660 that are pivotally pinned by corresponding pins 661 or are otherwise pivotally coupled to the wall 619 of the distal end of the handpiece 616.

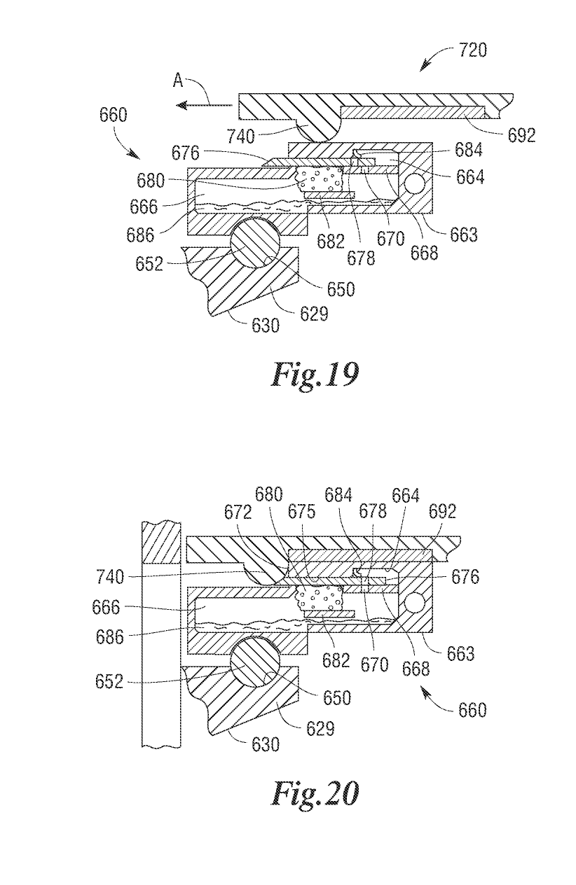

FIGS. 15-18 illustrate one form of a heat generating/cooling unit 660 of an embodiment of the present invention. In various embodiments, each heat generating/cooling unit 660 has a body portion 663 that may be fabricated from, for example, aluminum or engineered plastics such as polycarbonate, and be configured with an upper chamber area 664 and lower chamber area 666 therein that are separated by a wall 668 that has a fluid return passage 670 therethrough. The outer perimeter has a retention ledge 672 formed thereon for retaining engagement with a locking protrusion or protrusions 740 (FIGS. 19 and 20) formed in the shroud 720 of the surgical tool 700 as will be further discussed below. A return opening bar 676 slidably extends through a passage 675 in the body portion 663 defined by a sponge member 680 and the wall 668. The sponge member 680 may be supported on another wall portion 682 as shown. Return opening bar 676 has a hole 678 therethrough that may be coaxially aligned with the fluid return passage 670 to enable fluid/vapor to pass between the lower chamber 666 and the upper chamber 664. A bellows or wiper arrangement 684 may be provided in the upper chamber 664 for sliding engagement with the return opening bar 676 such that the bellows 684 serves to seal off the passage 675 when the return opening bar 676 has not been axially advanced into the upper chamber 664. A heating/cooling medium 686 is provided in the lower chamber 666. In various embodiments, the heating/cooling medium 686 may comprise, for example, a liquid that has a relatively low boiling point such as acetone.

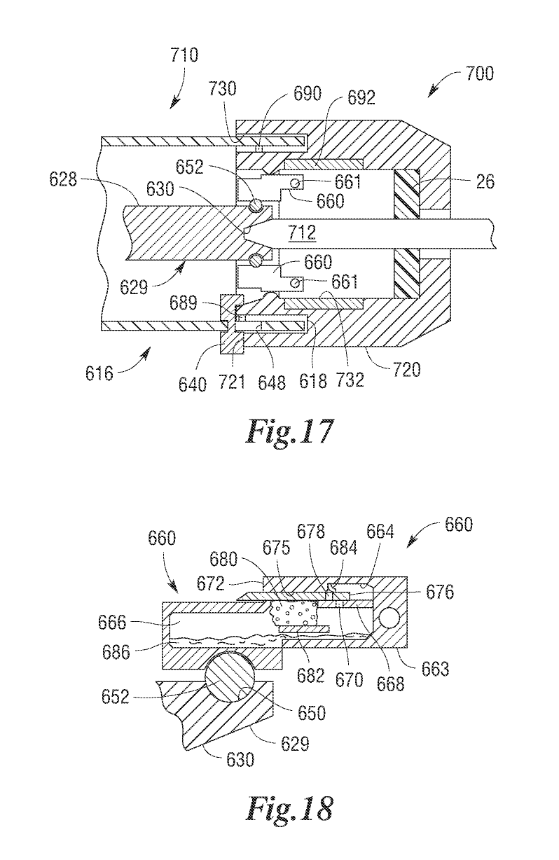

A method for coupling a surgical tool 700 to a handpiece 616 will now be described. FIG. 15 illustrates the positions of various components in the handpiece 616 and the surgical tool 700 prior to insertion of the distal end 618 of the handpiece into the shroud 720 of the surgical tool 700. To commence the coupling process, the clinician inserts the distal end 618 of the handpiece 616 into the shroud 720 of the surgical tool 700. See FIG. 16. At this point, the handpiece 616 and the surgical tool 720 are essentially at room temperature. As the distal end of the handpiece 618 enters the annular cavity 730 in the shroud 720, a power activation switch 690 mounted in the distal end portion 618 of the handpiece 616 permits current to flow to the generator to cause the transducers to be energized. In various embodiments, when the waveguide 628 and the locking ring 652 supported thereon are at room (neutral) temperature, the locking ring 652 is contracted about the distal end of the waveguide 628 such that the cavity 630 therein will not fully accept the frusto-conically shaped proximal end 712 of the surgical tool 700. However, activation of the transducers causes the waveguide 628 to heat the locking ring 652 causing it to expand to a point wherein the proximal end 712 of the surgical tool may be properly seated within the cavity 630. As the coupling process is initiated, the locking protrusion 740 (FIG. 19) pivots each of the heat generating/cooling units 660 about their respective pins into tight contact with the vibrating waveguide 628 to facilitate the generation of heat around the locking ring. This pivoting action is represented by arrows “A” in FIG. 16. When the proximal end 712 is completely seated within the cavity 630, the locking protrusion 740 snaps over the retention ledge 672 on the heat generating/cooling units 660 as shown in FIGS. 17 and 20. Those of ordinary skill in the art will appreciate that as the locking protrusion 740 snaps over the retention ledges 672, the clinician may be provided with tactile feedback and/or an audible click to indicate that the surgical tool 700 has been properly advanced to the coupled position. The locking protrusion 740 enables the heat generating/cooling units to pivot back to a neutral or unpivoted position wherein the locking protrusion 740 and the retention ledges still retain the surgical tool 700 in the coupled position. When in the coupled position as shown in FIGS. 17 and 20, the distal end 721 of the shroud 720 activates power deactivation switch 689 which stops the flow of electrical current to the transducers. The coupling procedure is now complete. The clinician is now free to use the system. It will be further understood that further operation of the transducers will cause the locking ring 652 to once again expand; however, the locking protrusion 740 and retention ledges 672 serve to maintain the coupled engagement between the surgical tool 700 and the handpiece 616.

Turning to FIGS. 18-20, it is desirable for the locking ring 652 to be hot during the initial coupling process to enable the proximal 712 end of the tool 700 to be inserted into the cavity 630. During that heating process, the liquid 686 resides in the sponge 680 and in the upper chamber 664. As can be seen in FIG. 20, when in the locked position, the heat generating cooling units 660 are adjacent a heat sink ring 692 mounted within the wall of the shroud 720. Such arrangement assists in dissipating the heat from the heat generating/cooling units 660. When the locking protrusion 740 is in the retention position (FIGS. 17 and 20) and the transducers have been deactivated, it is desirable for the locking ring 652 to cool to further secure the distal end of the waveguide 629 to the proximal end 712 of the tool 700. Advancement of the locking protrusion 740 to the locked position biases the return opening bar 676 to cause hole 678 in the bar 676 to be aligned with the return passage 670 to enable the fluid 686 in the upper chamber 664 to flow into the lower chamber 666. As the liquid 686 flows out of the upper chamber 684 it contacts the hot lower chamber 666 surfaces and evaporates to cool those surfaces and ultimately the locking ring 652.

To detach the surgical tool from the handpiece 616, the clinician moves the release ring 640 to activate the activation switch 689 or contact which causes the transducers to start the vibration process and begin the heating cycle. As the heat generating/cooling units 660 begin to heat up, the locking ring 652 begins to expand to enable the clinician to pull the surgical tool apart from the handpiece 616. When the parts have been separated, the power activation switch discontinues the power to the transducers after the power actuation switch is no longer activated by the distal end 721 of the tool shroud 720. Those of ordinary skill in the art will appreciate that a variety of known switches and switching arrangements, microprocessor controlled contacts, etc. may be used to activate and deactivate the transducers during the tool coupling process without departing from the spirit and scope of the present invention. For example, the power activation switches may comprise proximity sensing switches or contacts that are coupled to a microprocessor housed in or mounted adjacent to the generator.

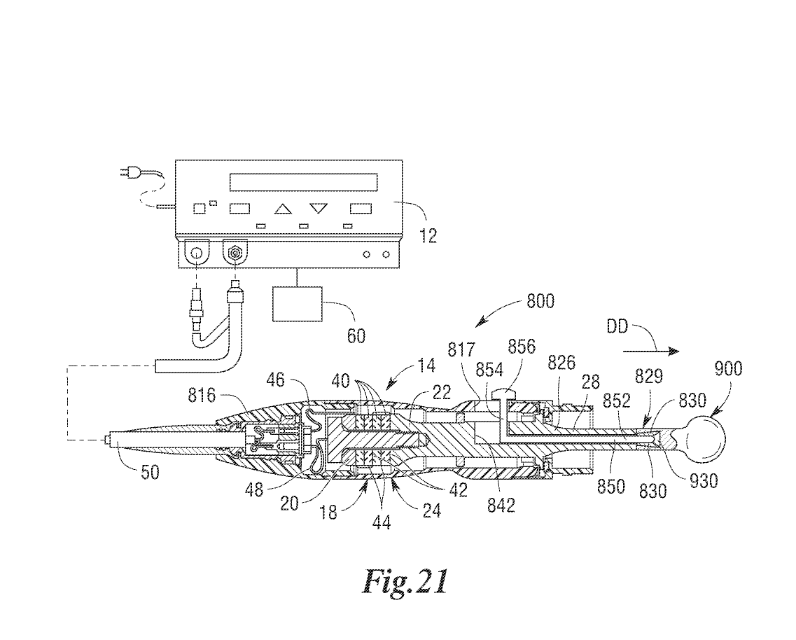

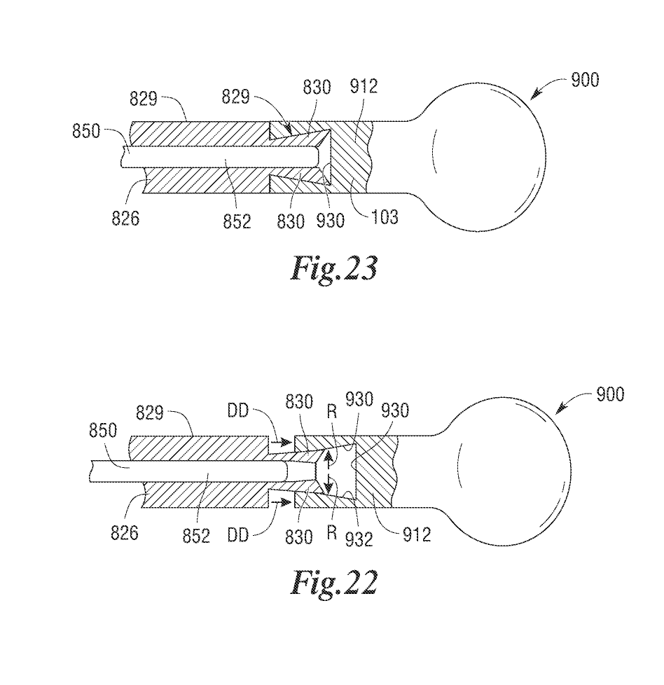

FIGS. 21-23 illustrate another surgical tool system 800 embodiment of the present invention that includes a generator 12 and a handpiece 816 that is substantially similar in design and construction as handpiece 16 described above, except for the differences noted below. For example, the distal end 829 of the waveguide 826 is selectively radially expandable to enable the distal end 829 to be effectively coupled to the proximal end 912 of the surgical tool 900. As can be most particularly seen in FIGS. 22 and 23, the distal end 829 of the waveguide 826 has two opposed lugs 830 that are shaped to retainingly engage a cavity 930 in the proximal end 912 of the surgical tool 900. The cavity 930 may be provided with tapered walls 932 such that when the lugs 830 are inserted in cavity 930 and then moved radially (arrows “R”), the lugs 830 serve to pull the tool 900 into retaining engagement with the distal end 829 of the waveguide 826 as shown in FIG. 23. In various embodiments, the waveguide 826 may be fabricated from, for example, aluminum 7075-T6.

Various embodiments may include an axially movable actuator rod 850 that is movably supported within a slot 840 in the waveguide 826. The actuator rod 850 may be fabricated from, for example, ultem, PEI and have a distal end 852 that is sized to extend between lugs 830 and, when advanced distally between the lugs 830, causes the lugs 830 to move radially. As can be seen in FIG. 21, the actuator rod 850 may have a radially extending portion 854 that extends through slots 842 and 817 in the waveguide 826 and handpiece 816, respectively. The radially extending portion 854 may terminate in a button portion 856 that facilitates actuation of the rod 850 by the clinician.

Thus, to couple the surgical tool 900 to the handpiece 816, the clinician inserts the lugs 830 into the cavity 930 while the actuator rod 850 is in an unactuated position (FIG. 22). Once the lugs 830 are inserted into the cavity 930, the clinician may slide the button portion 856 in the distal direction “DD” to cause the distal end 852 of the actuator rod 850 to axially move between the lugs 830 to cause the to move radially and engage the tapered walls of the cavity 930 (FIG. 23).

Various devices disclosed herein can be designed to be disposed of after a single use, or they can be designed to be used multiple times. In either case, however, the device may be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide or steam.

Although various embodiments have been described herein, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An ultrasonic surgical instrument having a handpiece operably supporting at least one ultrasonic transducer therein, said ultrasonic surgical instrument comprising:
a waveguide protruding distally from the handpiece and operably interacting with the at least one ultrasonic transducer; and
a surgical tool having a coupling portion configured to be coupled to a distal end of said waveguide such that upon application of thermal energy to one of said distal end of said waveguide or said coupling portion of said surgical tool, said surgical tool is coupled to said waveguide and, upon discontinuing the application of said thermal energy, said surgical tool may be detached from said waveguide.

2. The ultrasonic surgical instrument of claim 1 wherein said waveguide is fabricated from at least a first material such that at least a distal end portion of said waveguide has a first coefficient of thermal expansion and wherein said coupling portion of said surgical tool comprises a proximal end portion fabricated from at least a second material such that said proximal end portion of said surgical tool has a second coefficient of thermal expansion that is less than said first coefficient of thermal expansion, said proximal end portion of said surgical tool having a cavity therein for receiving said distal end portion of said waveguide therein, said cavity sized and shaped relative to said distal end portion of said waveguide such that upon application of said thermal energy to said distal end portion when said distal end portion is received in said cavity, said distal end portion expands to retain said distal end portion within said cavity.

3. The ultrasonic surgical instrument of claim 2 further comprising a heat applicator communicating with said waveguide.

4. The ultrasonic surgical instrument of claim 3 wherein said heat applicator selected from the group of heat applicators consisting essentially of: a radio frequency induction coil or a resistive thermoelectric heat element.

5. The ultrasonic surgical instrument of claim 2 wherein said cavity is in said distal end of said waveguide and is sized and shaped to receive said proximal end portion of said surgical tool therein and wherein said second coefficient of thermal expansion is greater than said first coefficient of thermal expansion.

6. The ultrasonic surgical instrument of claim 1 wherein said coupling arrangement comprises:
a proximal end portion configured to mate with said distal end of said waveguide; and
at least one selectively expandable latch member on said surgical tool configured to retainingly engage a corresponding portion of said handpiece to retain said distal end of said surgical tool in mating engagement with said distal end portion of said waveguide upon application of an actuation signal to said at least one selectively expandable latch member.

7. The ultrasonic surgical instrument of claim 6 wherein said corresponding portion of said handpiece comprises a tapered surface configured to interact with said at least one selectively expandable latch member such that, when said at least one selectively expandable latch member expands into contact with said tapered surface, said proximal end of said surgical tool is urged into mating engagement with said distal end portion of said waveguide.

8. The ultrasonic surgical instrument of claim 7 wherein said at least one selectively expandable latch member is mounted to a proximally extending shroud portion coupled to said surgical tool.

9. The ultrasonic surgical instrument of claim 8 further comprising:
at least one electrical contact supported in said proximally extending shroud portion and electrically coupled to said at least one expandable latch member; and
at least one activation contact supported on said handpiece and in electrical communication with a source of electrical current, said at least one activation contact oriented to engage said at least one electrical contact when said surgical tool is moved to a coupled position such that said electrical current is permitted to flow to said at least one selectively expandable latch member from said source of said electrical current.

10. The ultrasonic surgical instrument of claim 8 wherein said surgical tool may be decoupled from said distal end of said waveguide upon discontinuing the flow of electrical current to each of said at least one selectively expandable latch members.

11. The ultrasonic surgical instrument of claim 6 wherein each said at least one selectively expandable latch member is fabricated from a shape memory alloy.

12. An ultrasonic surgical instrument having a handpiece operably supporting at least one ultrasonic transducer therein, said ultrasonic surgical instrument comprising:
a waveguide protruding distally from the handpiece and operably interacting with the at least one ultrasonic transducer such that upon activation of said at least one ultrasonic transducer, said waveguide transmits thermal and vibratory energy to a distal end portion thereof, said distal end portion comprising a meltable alloy material; and
a surgical tool having a proximal end portion having a cavity sized to receive said distal end portion therein such that upon activation of said at least one ultrasonic transducer when said distal end portion is received within said cavity, said meltable alloy forms a welded connection between said surgical tool and said waveguide.

13. The ultrasonic surgical instrument of claim 12 wherein said at least one ultrasonic transducer is activated at a predetermined level of power for a predetermined length of time, wherein said predetermined level of power is greater than a normal power level used during operation of said surgical instrument.

14. An ultrasonic surgical instrument having a handpiece operably supporting at least one ultrasonic transducer therein, said surgical instrument comprising:
elongated means for transmitting ultrasonic energy from the at least one ultrasonic transducer;
a surgical tool; and
means for coupling said surgical tool to said elongated means upon application of a coupling signal to one of said elongated means and said surgical tool, said coupling means configured to permit said surgical tool to be decoupled from said elongated means when said application of said coupling signal is discontinued.

15. The ultrasonic surgical instrument of claim 14 wherein said coupling signal comprises the application of electrical current to at least one means for latching supported on one of said surgical tool and said elongated means.

16. The ultrasonic surgical instrument of claim 14 wherein said coupling signal comprises the application of thermal energy to one of said elongated means and said surgical tool.

17. An ultrasonic surgical instrument having a handpiece operably supporting at least one ultrasonic transducer therein, said ultrasonic surgical instrument comprising:
a waveguide protruding distally from the handpiece and operably interacting with the at least one ultrasonic transducer, said waveguide being fabricated from at least a first material such that at least a distal end portion of said waveguide has a first coefficient of thermal expansion; and
a surgical tool having a proximal end portion, said surgical tool fabricated from at least a second material such that at least the proximal end portion of said surgical tool has a second coefficient of thermal expansion that is less than said first coefficient of thermal expansion, said proximal end portion of said surgical tool having a cavity therein for receiving said distal end portion of said waveguide therein, said cavity sized and shaped relative to said distal end portion of said waveguide such that upon application of thermal energy to said distal end portion when said distal end portion is received in said cavity, said distal end portion expands to retain said distal end portion within said cavity.

18. A method for coupling a surgical tool to a waveguide of an ultrasonic surgical instrument that has a handpiece that operably supports at least one ultrasonic transducer therein that operably interacts with the waveguide, said method comprising:
positioning a proximal end of the surgical tool in mating contact with a distal end of the waveguide;
applying a coupling signal to one of the waveguide and the proximal end of the surgical tool to cause the proximal end of the surgical tool to be coupled to the distal end of the waveguide; and
discontinuing the application of the coupling signal to permit the surgical tool to be decoupled from the distal end of the waveguide.

19. The method of claim 18 wherein said applying a coupling signal comprises applying thermal energy to one of the waveguide and the surgical tool.

20. A method for coupling a surgical tool to a waveguide of an ultrasonic surgical instrument that has a housing that operably supports at least one ultrasonic transducer therein that operably interacts with the waveguide, said method comprising:
positioning a proximal end of the surgical tool in mating contact with a distal end of the waveguide; and
operating the at least one ultrasonic transducer at a predetermined power level that is greater than a normal operating power level for a period of time sufficient to cause at least a portion of the distal end of the waveguide to be welded to the proximal end of the surgical tool and thereafter operating the at least one ultrasonic transducer at the normal operating power to complete a surgical task with the surgical tool.

21. An ultrasonic surgical instrument having a handpiece operably supporting at least one ultrasonic transducer therein, said surgical instrument comprising:
a waveguide protruding distally from the handpiece and operably interacting with the at least one ultrasonic transducer, said waveguide having a distal end that has a tool-receiving cavity therein;
a surgical tool having a proximal end portion configured to be received within said tool-receiving cavity in said distal end of said waveguide upon application of thermal energy to said distal end and a shroud portion surrounding said proximal end and protruding proximally therefrom;
a coupling arrangement for selectively retaining the proximal end of the surgical tool within said tool-receiving cavity, said coupling arrangement comprising:
a locking member supported on said distal end of said waveguide adjacent said cavity, said locking member configured to expand from an unexpanded state to an expanded state when ultrasonic energy is applied to said waveguide from said at least one ultrasonic transducer and to return to the unexpanded state when said application of ultrasonic energy is discontinued; and
at least one heat generating and cooling unit movably coupled to said handpiece and interacting with said locking member, said at least one heat generating and cooling unit configured for locking engagement and disengagement with corresponding portions of said shroud.

22. The ultrasonic surgical instrument of claim 21 wherein when said locking member is in said unexpanded state, said proximal end of said surgical tool cannot be seated within said tool-receiving cavity.

23. The ultrasonic surgical instrument of claim 21 wherein each of said at least one heat generating and cooling units is pivotally coupled to said handpiece and is configured to pivot into frictional contact with said locking member as said corresponding portions of said shroud are brought into axial mounting engagement with said at least one heat generating and cooling units.

24. The ultrasonic surgical instrument of claim 21 wherein said at least one heat generating and cooling units each contain an evaporative liquid.
